(19) United States
(12) Patent Application Publication
(10) Pub. No.: US 2008/0292046 A1
(43) Pub. Date: Nov. 27, 2008

(54) Bronchopulmonary medical services system and imaging method

(76) Inventors: Estelle Camus, Mountain View, CA (US); Oliver Meissner, Munchen (DE); Martin Ostermeier, Buckenhof (DE); Thomas Redel, Poxdorf (DE)

Correspondence Address: SIEMENS CORPORATION;INTELLECTUAL PROPERTY DEPARTMENT, 170 WOOD AVENUE SOUTH, ISELIN, NJ, 08830, US

(21) Appl. No.: 12/150,581

(22) Filed: Apr. 29, 2008

Related U.S. Application Data
(60) Provisional application No. 60/928,291, filed on May 9, 2007.

(30) Foreign Application Priority Data
May 9, 2007 (DE) 10 2007 021 717.1

(51) Int. Cl. A61B 6/00; A61B 5/05

(52) U.S. Cl. 378/4; 600/427

(57) ABSTRACT

A bronchopulmonary medical services system is provided in order to offer medical services to a patient in a single location. In one aspect, the system is provided with a patient alignment device and a C-arm imaging device. A medical services suite may be equipped with various medical service devices such that the patient receives medical services in a single location.

### CROSS REFERENCE TO RELATED APPLICATIONS

The present application claims the priority of the provisional patent application filed on May 9, 2007, and assigned application No. 60/928,291, to the German application No. 10 2007 021 717.1 filed on May 9, 2007, and to the U.S. application Ser. No. 11/977,465 filed on Oct. 25, 2007, all of the applications are incorporated by reference herein in their entirety.

### FIELD OF THE INVENTION

The present invention relates to a bronchopulmonary medical services system and an imaging method.

### BACKGROUND OF THE INVENTION

Bronchopulmonary damage covers an inhomogeneous group of different diseases. Among these diseases are functional, inflammatory and cancerous diseases of the tracheobroncial tract, the lung, as well as the associated fibrous tissues, vascular systems, and lymphatic systems. The diagnosis and/or therapy of a patient suspected of bronchopulmonary damage may involve several methods which is time-consuming and often complex. Such methods may include for, example, X-ray imaging, computerized tomography (CT) scans, bronchoscopy, and bronchoveolar lavage.

### SUMMARY OF THE INVENTION

The term “medical services” is used herein to refer to pre-treatment activities such as diagnostics, treatment activities such as the removal of tissue, or post treatment activities such as monitoring the condition of a patient after treatment as well as any combination of pre-treatment, treatment, and post-treatment. The term “therapy” is used herein to refer to treatment activities.

In one aspect, a method for providing bronchopulmonary medical services to a patient at a single location of a medical services suite is provided. The patient is positioned via an alignment device. A medical service is provided to the positioned patient whereby an image is produced via an X-ray imaging system having multi-axis movement in order to vary position of the patient image. A determination is made from the image whether a subsequent medical service is to be provided to the patient and if so the subsequent service is provide. The process of the determination and provision of the subsequent medical service is repeated until the determination to not provide further services. In one aspect the determination is made based on the previously medical service. Other aspects may involve patient condition and or a combination of the medical services.

The subsequent medical service may be a subsequent image of the patient. The subsequent image may include an image such as an angiographic image, intra-aterial image angiography image, bronchoscopic image, morphology image, and functional image. A contrast may be applied prior to the image and/or subsequent image.

The patient may be repositioned in accordance with the subsequent medical service. Additionally the patient may be transferred to the suite and/or to the alignment device prior to the positioning the patient. Also the patient may be transferring from the alignment device and/or suite after a determination to not provide the subsequent medical service. In one aspect at least part of the transfer is via a robot. The robot may be incorporated within the alignment device.

Various subsequent medical service may be provided such as a bronchosopic procedure or a percutaneous procedure. To this end, the X-ray imaging device may comprise a C-arm angiography device or a C-arm CT angiography device. An endoscopic bronchoscopy device may be spatially combined to the X-ray imaging device. Also a percutaneous intervention device may be spatially combined to the X-ray imaging device.

The percutaneous intervention device may be an image-guided thermal ablation device, a radiation therapy device, a cryotherapy device, a robotic biopsy device, a robotic puncture device or combinations thereof. The endoscopic bronchoscopy device may provide services such as a biopsy, cauterization, local-thermo, chemotherapy, bronchoalveolar lavage, stent insertion, removal of an object lodged in an airway of the patient.

The X-ray image may be recorded and spatially combined to the endoscopic bronchoscopy device. A recording of a bronchoscopic image of the patient via a navigation may be provided. Various navigation methods may be used such as 2D, 3D online fluoroscopic imaging guidance, a prerecorded magnetic, optical, and/or ultrasound positioning navigation. In addition navigation may be performed by a combination or a superimposition of an endobronchial ultrasound measurement, an optical coherence tomography measurement, an fluorescent measurement, or an molecular imaging measurement.

### BRIEF DESCRIPTION OF THE DRAWINGS

The following describes the invention using one example of an embodiment with reference to the drawings, in which:

FIG. 1 shows a schematic view of a bronchopulmonary diagnostic and/or therapeutic system according to the invention, and

FIG. 2 shows a flow diagram representing the work flow with the imaging method according to the invention.

### DETAILED DESCRIPTION OF THE INVENTION

Typically a conventional radiography of the thorax is used first in order to obtain an X-ray image. X-ray images via the ubiquitous conventional radiography devices are relatively inexpensive. However, such X-ray images have limitations with regard to their resolution and visualization due to, for example, the overlay of different organ systems. This especially impedes the evaluation of intrapulmonary nodules and the visualization of subtle opacifications of the lung parenchyma.

High-resolution computerized tomography (CT) scanners have provided great advances with respect to resolution and imaging in the form of sectional views or three-dimensional representations. Different windowing levels may be used to evaluate different tissue types and organ systems. Additionally, with the use of intravascular contrast material, clear differentiation between cardiovascular structures, lymph nodes and lung parenchyma are possible. However, even with the high-resolution CT scanners, it is often not possible to differentiate between inflammatory diseases and diffuse-cancerous infiltrates or fibrous tissue changes.

Bronchoscopy is a medical procedure where a tube is inserted into the airways, usually through the nose or mouth. This allows for the examination inside a patient's airway for abnormalities such as foreign bodies, bleeding, tumors, or inflammation in addition to providing the ability to perform a tissue biopsy. Bronchoscopy, which commonly uses a fluoroscopic or CT guidance, may be performed using a rigid bronchoscope or a flexible bronchoscope. A flexible bronchoscope includes optical fibers (fiber optics) that transmit light images as the tube bends. The rigid bronchoscope is a straight, hollow, metal tube and performed under general anesthesia. Since bronchoscopy is invasive, albeit minimally invasive, it may be preferable to reserve this method when results from the non invasive methods such as X-Ray and CT scan are inconclusive. Additionally, therapies such as removing objects lodged in the airway, stent insertion, cauterization may be performed during bronchoscopy.

Further diagnostics using bronchoscopy may be achieved by injecting sterile fluid into the lungs and removing the fluid. The removed contains secretions, cells and protein from the lower respiratory tract and can be analyzed to access possible lung diseases. This type of procedure is revered to as bronchoalveolar lavage (BAL).

Conventionally, the methods for different medical services take place in different locations, which may be different institutes in one hospital, different laboratories or the like. This is time-consuming, arduous for the patient and does not enable the individual imaging and diagnostic steps to be directly linked to each other. Furthermore, different imaging techniques are often performed by different institutions. Substantial problems might occur with the co-registration of the different imaging leading to inaccuracy. For example, overlaying the different images may be useful in diagnostics or navigation in invasive procedures. However, it may be difficult to impossible to overlaying the different images obtained via the different techniques resulting in inaccuracies such as nodule location, or guidance of minimally-invasive procedures.

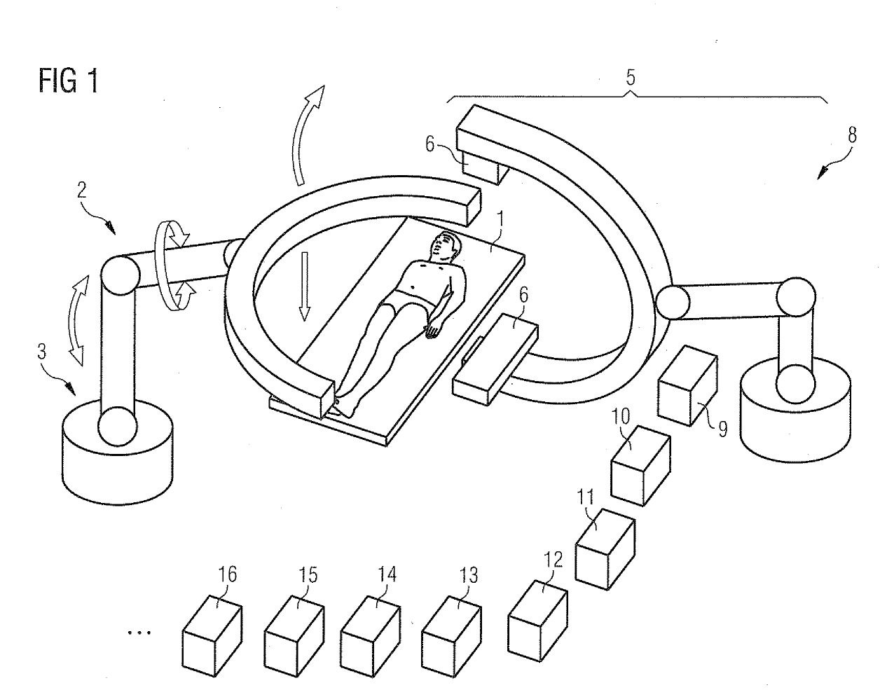

Referring to FIG. 1 a schematic view of an embodiment of a bronchopulmonary medical services suite 10 according to the present invention is shown. The medical services suite 10 includes a patient alignment device 20 and a C-arm imaging device 30.

The patient alignment device 20 includes a patient support apparatus 22 and a robotic adjustment unit 24. The support apparatus 22, which accommodates a patient 50 during at least a portion of the medical services, may be a stretcher, gurney or the like. The support apparatus 22 may be permanently attached to adjustment unit 24. Alternately, the support apparatus 22 may be removably attached to the adjustment unit 24.

The adjustment unit 24 adjusts the support apparatus 22 thereby adjusting the patient 50. Thus, an optimum alignment of the patient 50 may be achieved according to the medical services and/or condition of the patient. For example, it may be beneficial when the patient 50 suffers from dyspnea or a blockage of the tracheobronchial tree to adjust the support apparatus 22, with the patent secured to the support apparatus 22, into a vertical position. The adjustment unit 24 is not limited to an adjustment of the support apparatus 22 along one axis but may include adjustments along multiple axis as well as a rotational adjustment. For example, the adjustment unit 24 may adjust the height of the support apparatus 22.

In the embodiment shown in FIG. 1, the adjustment unit 24 is a robot. However, other devices may be used such that the adjustment unit 24 may be controlled manually, remotely, electronically, via a computer or the like.

The C-arm imaging device 30 includes a C-arm support 32 to which an X-ray source 34, which may include a diaphragm to limit the field of view, and an X-ray detector 36 may be mounted so as to face each other along a central axis of radiation. The C-arm support is mounted to an adjustment device 38.

In the embodiment shown in FIG. 1, the adjustment device 38 is mounted to a wall and is capable of moving in longitudinal and traverse directions with respect to the mounting device. However, the adjustment device 38 may be mounted to a different surface such as the ceiling or the floor or may be free standing without being mounted. Furthermore, the adjustment device 38 provide for movement in other directions including a rotational movement. Thus, the adjustment device 38 may have a multi-axis degree of freedom. The adjustment device 38 may be a robot as shown in FIG. 1 or other device which may be controlled manually, remotely, electronically, via a computer or the like.

The patient alignment device 20 may communicate with the C-arm imaging device 30 via wires or in a wireless manner whereby the alignments provided by the adjustment unit 24 and the adjustment device 38 may be coordinated. In a further embodiment, the communication between alignment device 20 and the C-arm imaging device 30 may be via an intermediate control device.

The medical services suite 10 may also be equipped with a bronchoscopic device, an intrabronchial or intravascular device (ultrasound, optical), an opto-magnetic navigation device for automatic/semi-automatic medical services guidance, or PET/SPECT imaging.

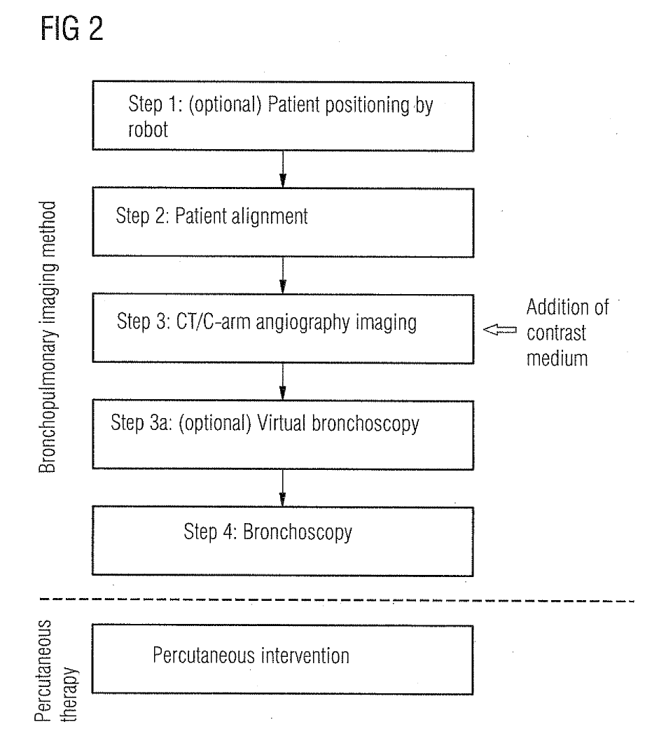

Referring now to FIGS. 1 and 2 an embodiment of a work flow for bronchopulmonary medical services is described. The patient 50 is transferred to the support apparatus 22 for medical services of a possible bronchopulmonary disease 200. The transfer may be manually or via a robotic system. The position of the patient 50 is adjusted via the adjustment unit 24 with consideration of the condition of the patient and the medical services to be provided 202.

A determination is made if the imaging will require a contrast agent 204. If so the contrast is administered prior to obtaining the image 206. An imaging device 30 performs a scan of the thorax in order to obtain the image 208. A determination is made if more imaging is required 210. If more imaging is required the flow is redirected to the positioning of the patient. The contrast material can either be delivered vascular, orally or tracheobronchial.

The image obtained may include 2d and/or 3d as well as be a morphology and/or functional image. Additionally, the imaging may include angiographic, intra-aterial angiography, and bronchoscopic images. Angiographic imaging may be used, for example, to view the intrathoracic vessels and the heart. Intra-arterial angiography may be used for example when arterial/venous malformation is suspected or to better detect the vascularization of a tumor.

After the images are obtained a determination is made if medical services should continue 212. Medical services is performed when it is determined that the medical services should continue 214. The medical services may be a bronchoscopic procedure or a percutaneous procedure.

In the case of a bronchoscopic procedure, a biopsy, cauterization, local-thermo and/or chemotherapy may be performed. The bronchoscopy may be navigated via the previously acquired imaging which is reconstructed having a 2d and/or 3d display. Guidance may also include magneto-optical navigation.

This can be followed by percutaneous therapy with percutaneous intervention devices The bronchopulmonary diagnostic and therapeutic system allows percutation/intervention devices in the immediate spatial vicinity of the imaging devices. Such percutation/intervention devices include, for example, an image-guided thermal ablation device, a robotic biopsy device, a robotic puncture device, a radiation therapy device and/or cryotherapy devices. The robotic biopsy and puncture devices may be lightweight. For example, the robotic device may be portable.

There is no need for laborious bed-changing or to subject the patient to the stress of transferal to different examination rooms. In addition, 3D reconstructions of the C-arm CT images and 2D-3D overlay of cross-sectional and fluoroscopic images provide a particularly good basis for diagnostic and/or therapeutic bronchoscopy. Rapid decisions regarding further therapeutic measures can be taken on the basis of the examination using the coherent diagnostic and therapeutic system according to the invention. Furthermore, it is anticipated that providing the therapy in a single location, also referred as a one stop therapy, would yield a cost savings.

The invention covers all variants which fall within the sphere of protection of the attached claims and the above description of an example of an embodiment is for illustrative purposes only.

1. A method for providing bronchopulmonary medical services to a patient at a single location of a medical services suite, comprising:
positioning the patient via an alignment device;
providing a medical service by producing an image of the positioned patient, the image produced via an X-ray imaging system having multi-axis movement in order to vary position of the patient image;
determining from the previously provided medical service whether a subsequent medical service is to be provided to the patient;
providing the subsequent medical service to the patient when the determination is to provide the subsequent medical service, the subsequent medical service becoming the previously provided medical service; and
repeating the determining and the providing of the subsequent medical service until a determination to not provide the subsequent medical service.

2. The method as claimed in claim 1, wherein the image is a reconstructed 3D image of a region of interest of the patient.

3. The method as claimed in claim 1, wherein the image is a reconstructed combination of 2d AND 3D image of a region of interest of the patient.

4. The method as claimed in claim 1, wherein the subsequent medical service is a subsequent image of the patient.

5. The method as claimed in claim 4, further comprising applying a contrast prior to producing the image or the subsequent image is produced

6. The method as claimed in claim 4, wherein the subsequent image selected from the group consisting of angiographic, intra-arterial angiography, bronchoscopic, morphology, and functional.

7. The method as claimed in claim 1, further comprising repositioning the patient in accordance with the subsequent medical service.

8. The method as claimed in claim 1, further comprising transferring the patient via the alignment device to the positioning the patient and transferring the patient from the alignment device after a determination to not provide the subsequent medical service.

9. The method as claimed in claim 8, wherein the transfer is via a robot.

10. The method as claimed in claim 1, wherein the subsequent medical service is a bronchoscopic procedure or a percutaneous procedure.

11. The method as claimed in claim 1, wherein the X-ray imaging device comprises a C-arm angiography device or a C-arm CT angiography device.

12. The method as claimed in claim 1, wherein an endoscopic bronchoscopy device is spatially combined to the X-ray imaging device.

13. The method as claimed in claim 12, wherein the subsequent medical service is selected from the group consisting of subsequent image, biopsy, cauterization, local-thermo, chemotherapy, bronchoalveolar lavage, stent insertion, removal of an object lodged in an airway of the patient.

14. The method as claimed in claim 12, further comprising:
recording the X-ray image;
spatially combining the endoscopic bronchoscopy device to the X-ray imaging arrangement; and
recording an bronchoscopic image of the patient via a navigation.

15. The method as claimed in claim 14, wherein the navigation is performed by a 2D or 3D online fluoroscopic imaging guidance.

16. The method as claimed in claim 14, wherein the navigation is performed by a prerecorded magnetic, optical, or ultrasound positioning navigation.

17. The method as claimed in claim 14, wherein the navigation is performed by a combination or a superimposition of an endobronchial ultrasound measurement, an optical coherence tomography measurement, a fluorescent measurement, or a molecular imaging measurement.

18. The method as claimed in claim 1, wherein a percutaneous intervention device is spatially combined to the X-ray imaging device.

19. The method as claimed in claim 18, wherein the percutaneous intervention device is selected from a device selected from group consisting of: an image-guided thermal ablation device, a radiation therapy device, a cryotherapy, a robotic biopsy device and a robotic puncture device.

20. The method as claimed in claim 1, wherein the imaging system performs a preliminary virtual bronchoscopy of a patient.
